(19) United States
(12) Patent Application Publication
(10) Pub. No.: US 2022/0343999 A1
(43) Pub. Date: Oct. 27, 2022

(54) MOLECULAR PHENOTYPE CLASSIFICATION

(71) Applicant: UNIVERSITY OF SOUTHAMPTON, Southampton (GB)

(72) Inventors: James Philip Robertson SCHOFIELD, Southampton (GB); Fabio STRAZZERI, Southampton (GB)

(21) Appl. No.: 17/754,103

(22) PCT Filed: Sep. 22, 2020
(86) PCT No.: PCT/GB2020/052288

§ 371 Date: Mar. 23, 2022

(30) Foreign Application Priority Data
Sep. 23, 2019 (GB) 1913690.2

(51) Int. Cl. G16B 5/00

(52) U.S. Cl. CPC G16B 5/00

(57) ABSTRACT

Methods, systems, apparatuses and computer readable media are provided for characterizing a molecular phenotype of a biological sample using a biological interaction network. A biological interaction network includes a plurality of nodes, each node associated with a corresponding gene or protein. A method includes associating, with each node of the biological interaction network, a corresponding differential abundance value for the gene or protein to which that node corresponds, the differential abundance value derived from a comparison of a representative abundance value for the gene or protein in a biological sample exhibiting the molecular phenotype and a reference abundance value for the gene or protein. The method includes, using the differential abundance values of the nodes of the biological interaction network, performing a hill-climbing algorithm to partition the biological interaction network into clusters. The method includes determining, from the topology of the clusters, a signature of the molecular phenotype.

### TECHNICAL FIELD

The present disclosure relates to methods, computer-readable media, apparatuses and systems for characterising a molecular phenotype of a biological sample. The present disclosure further relates to methods, computer-readable media, apparatuses and systems for determining a molecular phenotype of a biological sample.

### BACKGROUND

Many drug trials routinely fail because they are not targeted at patients with the correct molecular biology for which a given drug would be most effective. For drugs already on the market, response to treatment is often variable, largely due to poor targeting of drugs to subtypes of disease. For example, a disease may comprise common symptoms from, for example, five different molecular dysfunctions (five different disease subtypes or disease phenotypes), but all five molecular dysfunctions may be treated by a therapy that only targets genes responsible for one of the five subtypes, leading to only a 20% success rate in treatment of these patients. This has large implications in terms of health, wellbeing and monetary costs.

Biomarkers can be used to identify patients with known disease phenotypes and enable recruitment of patients likely to respond to a specific drug, thereby increasing the power of the drug trial, enabling robust testing and increasing the probability of succeeding in bringing the drug to market. However, the large redundancy and variation in, for example, gene expression data means that standard statistical comparisons of gene expression can identify biomarkers with a poor success rate.

Personalised medicine enables efficient treatment of patients, by targeting therapeutics to patients with recognisable molecular phenotypes and requires the diagnosis of patients by biomarker expression. However, disease biomarkers rarely work in identifying patients for specific treatments. This is due to the oversimplification of the molecular signatures of disease by looking for the most differentially expressed genes between disease and control populations.

The present invention addresses at least some of these issues.

### SUMMARY

According to an aspect of the invention, a computer-implemented method is provided, the computer-implemented method for characterising a molecular phenotype of a biological sample using a biological interaction network. The biological interaction network comprises a plurality of nodes, each node associated with a corresponding gene or protein. The biological interaction network further comprises a plurality of edges, each edge connecting a pair of nodes and indicative of an interaction between the genes or proteins to which each node of that associated pair of nodes corresponds. The method comprises associating, with each node of the biological interaction network, a corresponding differential abundance value for the gene or protein to which that node corresponds. The differential abundance value is derived from a comparison of a representative abundance value for the gene or protein in a biological sample exhibiting the molecular phenotype and a reference abundance value for the gene or protein. The method further comprises, using the differential abundance values of the nodes of the biological interaction network, performing a hill-climbing algorithm to partition the biological interaction network into clusters. The method further comprises determining, from the topology of the clusters, a signature of the molecular phenotype.

As an example, consider gene expressions information and the diagnosis of disease states. The change in the expression of one gene impacts the expression of many other genes, some directly or indirectly cause disease symptoms, and some have no symptomatic effect. A disease phenotype may originate from one faulty element in a molecular pathway, but disease symptoms may arise from effects reverberating through gene pathways. A typical microarray of gene expression measures the expression of >50,000 genes, and even with FDR correction, any study looking at the difference in expression of individual genes is likely to identify false positive biomarkers of disease. Increasingly, biomarker panels are being developed, which have improved efficacy in distinguishing patients with different disease subtypes. However, most gene expression data is being ignored in traditional biomarkers.

Global gene expression data can provide a detailed picture of a molecular phenotype. Although each molecule may be measured with low accuracy, topological measurement of shape works like biology—the data points are noisy, high dimensional and with a lot of redundancy built in. A method such as the method for characterising a molecular phenotype described above uses, for example, biological pathways as coordinates for measuring the shape of global gene expression, accurately characterizing molecular phenotypes. Accordingly, by characterising a molecular phenotype using a method as described herein, the molecular phenotype can be identified using a topology-based signature which can be easily compared with new samples to identify whether those biological samples exhibit the molecular phenotype. A disease or disease-subtype has a common shape of gene expression relative to health, and these shapes can be identified. By mapping the differential abundance values (for example the differential gene expression values) of a patient relative to health on a gene network/pathway, the measurement of differential modulation of genetic pathways is enabled, and the identities and magnitudes of activated/inactivated pathways help to provide the “shape” of the disease.

Biological samples may come from any suitable source, for example from blood samples, tissue samples, cell samples and so on. Information on abundance values may be obtained using any suitable means, for example RNA microarrays, RNAseq, mass spectrometry or protein microarrays.

A biological interaction network may be any suitable network that applies to a biological system, for which the nodes of the network may be taken to represent genes or proteins and the edges can represent interactions between the genes or proteins. For example, a biological interaction network may comprise a protein-protein interaction network in which each node represents a protein and the interaction between the proteins are represented by the edges of the network. For example, the biological interaction network may comprise a gene regulatory network, or a gene co-expression network. A biological interaction network may represent a biological pathway. A biological interaction network may comprise a metabolomic network.

A molecular phenotype is a molecular characteristic resulting in a biological behaviour. A molecular phenotype may comprise, for example, a disease state. Characterising a molecular phenotype of a biological state may be understood to mean deriving some form of classifier or signature or identifier that can indicate from gene or protein abundance data a phenotype or state of the biological sample.

An “abundance value” as used herein may be understood to mean a value representative of an extent to which the gene or protein to which the abundance value corresponds is expressed in a sample. For example, an abundance value may comprise a gene expression value. A representative abundance value for a gene or protein in a biological sample exhibiting a molecular phenotype may comprise, for example, an average abundance value for the gene or protein from measurements of several samples exhibiting the molecular phenotype. Similarly, a reference abundance value for a gene or protein in a biological sample may comprise, for example, an average abundance value for the gene or protein from measurements of many samples taken from a cross-section of the population or taken from a cross-section of a known healthy population. The reference abundance value/control abundance value may therefore be thought of as the abundance value in the “average person”, or in the “average healthy person” as circumstances permit. The representative abundance value may be thought of as the abundance value in the “average patient having the molecular phenotype”. Of course, the skilled person would appreciate that the reference abundance values and the representative abundance values may be derived in other ways.

A differential abundance value (which in some examples may be a differential gene expression value) may be derived from a comparison of the representative abundance value and the reference abundance value. Accordingly, the differential abundance value represents which genes or proteins represented in the biological interaction network are up-regulated or down-regulated with respect to the reference. A logarithmic scale may be used for the differential abundance value. The method may be concerned primarily with fold-change values.

A hill-climbing algorithm is a technique in numerical analysis for optimizing a target function in an iterative manner. In what follows, the inventors have used their own invented Morse Theory algorithm, but any type of hill-climbing algorithm may be suitable.

Partitioning the biological interaction network into clusters may be understood to mean identifying, for example, that a first set of nodes of the biological interaction network belong to a first cluster, while a second set of nodes of the biological interaction network belong to a second cluster, disjoint from the first cluster.

Using the differential abundance values of the nodes of the biological interaction network to perform a hill-climbing algorithm to partition the biological interaction network into clusters may comprise, for example, determining scores for nodes of the network based on the differential abundance values and optionally based on weights of the edges between nodes.

A cluster may be understood to be a subgraph of the original biological interaction network, disjoint from other subgraphs of the network. A cluster may represent, for example, a biological pathway/sub-pathway that is of relevance to the molecular phenotype.

The term “signature” as used herein is to be understood broadly. The signature may be determined from, for example, a size of the largest cluster, the number of clusters, the underlying pathways/subpathways relating to the clusters and so on. Other known measures of directed graphs can be used to determine a signature, for example the local information of a cluster, the global efficiency of a cluster, local efficiency of a cluster, or node degree of a cluster. The signature may or may not be unique.

Loosely speaking, a method for characterising a molecular phenotype of a biological sample described herein (and as described in more detail in relation to FIG. 2) may comprise hopping from node to node in the network and identifying, for each given node, a neighbouring node to which that given node “must” be connected in a cluster, based on the differential abundance values of that given node and the neighbouring nodes. That is, the identified necessary connection is in some way “important”. Having formulated such a “list” of important connections between nodes, a determination can accordingly be made that a first set of nodes of the biological interaction network are surely part of a first cluster, that a second set of nodes of the biological interaction network are surely part of a second cluster, and so on. Neighbouring nodes of each cluster may additionally be connected by “unimportant” edges i.e. edges that were not deemed to be “important”. Every node of the cluster is connected to every other node of that cluster either directly by an “important” edge, or indirectly by a chain of two or more “important” edges. If two nodes are not connected, directly or indirectly, by one or more “important” edges, then the two nodes may be considered to belong to different clusters. In short, after having performed the node hopping, a node in the first cluster will not be connected, directly or indirectly, to any node of a second cluster, by one of these “must-have” connections determined from the differential abundance values of nodes. The skilled person would of course appreciate that this description of how the method functions is for illustrative purposes only. The differential abundance values thus have a large influence over the clusters that emerge. As explained above, a differential abundance value for a gene or protein is derived from a comparison of a representative abundance value for the gene or protein in a biological sample exhibiting the molecular phenotype and a reference abundance value for the gene or protein.

If the reference values and representative values are derived from similar samples then one can expect this to be reflected in the resulting clusterings. If, for example, the biological sample exhibiting the phenotype is healthy and the reference abundance values are derived from e.g. an average healthy patient, then one would expect the underlying abundance value data for the healthy sample to be similar to the reference values, and this would be reflected in the differential abundance values. Accordingly, when performing the hill-climbing algorithm to partition the biological interaction network into clusters, the similarity between the representative values and the reference values would be demonstrated in the resulting clusters. In particular, the biological interaction network would usually be broken into a small number of large clusters, or may not even break into clusters at all.

Similarly, if the reference abundance values for the genes proteins were derived from one or more samples exhibiting a disease state, and the representative abundance values were derived from one or more samples exhibiting that same disease state, then the biological interaction network would usually be broken down into a small number of large clusters, or may not break into clusters at all. That is, the similarity of the representative sample and the reference sample is reflected in the size and shape of the resulting clusters.

In contrast, if the reference abundance values and the representative abundance values differ greatly for some genes or proteins, then this would be reflected in the resulting clusters.

For example, if the representative abundance values were derived from one or more samples exhibiting a particular disease state while the reference abundance values were derived from data for one or more healthy samples/samples not exhibiting that disease state, then one would expect that for some genes or proteins of the biological interaction network there would be a significant discrepancy between the representative abundance value for that gene or protein and the reference abundance value for that gene or protein, and this would be reflected in the differential abundance value for that gene or protein. When performing the hill-climbing algorithm, it is therefore much more likely that the biological interaction network will be broken into several, smaller clusters, than it would have been had the representative values represented a healthy sample (similar to the reference).

The size and topology of the clusters may be used to determine a signature for a molecular phenotype of interest. For example, the signature may be determined from the size of the largest cluster, or from some other function of the sizes of the clusters.

Further advantageously, as the clusters represent underlying subgraphs of the biological interaction network, they may be used to identify which underlying pathways/subpathways are most influenced by e.g. a disease. For example, if the representative abundance values are derived for a healthy sample and the reference values are derived for a healthy sample, then then one or more large clusters would emerge, indicating that the sample was healthy. In contrast, if the representative abundance values reflected a disease state, then the resulting clusters would be smaller and more plentiful. As the clustering process is influenced greatly by the underlying differential abundance values for nodes of the biological interaction network, these smaller clusters indicate that at least one gene or protein of associated with that cluster is having an undue influence (compared to a healthy sample) on the other genes or proteins in that cluster. Accordingly, differences in pathway modulation between molecular phenotypes can be clearly distinguished, by measuring the difference in network topology of gene/protein expression annotated on a biological interaction network.

The method may further comprise associating, with each edge of the plurality of edges, a weight. Performing the hill-climbing algorithm may comprise performing the hill-climbing algorithm using the weights of the edges.

Each node of the plurality of nodes may be associated with a corresponding gene. The representative abundance value for the gene may comprise a gene expression value for the gene. The reference abundance value for the gene may comprise a reference gene expression value for the gene. The differential abundance value may comprise a differential gene expression value, the differential gene expression value derived from a comparison of the representative gene expression value and the reference gene expression value.

The molecular phenotype of the biological sample may comprise a disease state of the biological sample.

The biological network may comprise a biological pathway, for example, a gene expression network.

Prior to the associating, the method may further comprise receiving data representative of the biological interaction network.

The method may further comprise, for each node of the biological network, receiving or determining the corresponding differential abundance value.

The reference abundance value for each node may comprise an average of abundance values for a plurality of biological samples. The reference abundance value for each node may be derived from, for example, a plurality of healthy biological samples. The reference abundance value for each node may be derived from, for example, a plurality of biological samples exhibiting some particular molecular phenotype such as a disease state.

The representative abundance value for each node may comprise an average of abundance values for a plurality of biological samples exhibiting the molecular phenotype. The representative abundance value for each node may be derived from, for example, a plurality of healthy biological samples (molecular phenotype here being understood to mean healthy). The reference abundance value for each node may be derived from, for example, a plurality of biological samples exhibiting some particular molecular phenotype such as a disease state.

Performing the hill-climbing algorithm may comprise performing a Morse theory algorithm.

Performing the hill-climbing algorithm to partition the biological interaction network into clusters may comprise, for each node of the biological interaction network, determining, for each neighbouring node of all neighbouring nodes connected to the node, a score based on the differential abundance value of that neighbouring node; determining the neighbouring node associated with the highest or lowest score; and determining that the node and the neighbouring node associated with the highest or lowest score are of the same cluster.

According to an aspect of the invention, a computer-readable medium is provided. The computer-readable medium has instructions stored thereon which, when executed by one or more processors, causes a method as described herein to be performed.

According to an aspect of the invention, an apparatus is provided, the apparatus for characterising a molecular phenotype of a biological sample. The apparatus comprises one or more memory devices configured to store a biological interaction network. The biological interaction network comprises a plurality of nodes, each node associated with a corresponding gene or protein. The biological interaction network further comprises a plurality of edges, each edge connecting a pair of nodes and indicative of an interaction between the genes or proteins to which each node of that associated pair of nodes corresponds. The apparatus comprises one or more processors. The one or more processors are configured to associate, with each node of the biological interaction network, a corresponding differential abundance value for the gene or protein to which that node corresponds, the differential abundance value derived from a comparison of a representative abundance value for the gene or protein in a biological sample exhibiting the molecular phenotype and a reference abundance value for the gene or protein. The one or more processors are further configured to, using the differential abundance values of the nodes of the biological interaction network, perform a hill-climbing algorithm to partition the biological interaction network into clusters. The one or more processors are further configured to determine, from the topology of the clusters, a signature of the molecular phenotype.

According to an aspect of the invention, a computer-implemented method is provided for determining a molecular phenotype of a biological sample using a biological interaction network. The biological interaction network comprises a plurality of nodes, each node associated with a corresponding gene or protein. The biological interaction network further comprise a plurality of edges, each edge connecting a pair of nodes and indicative of an interaction between the genes or proteins to which each node of that associated pair of nodes corresponds. The method comprises associating, with each node of the biological interaction network, a corresponding differential abundance value for the gene or protein to which that node corresponds, the differential abundance value derived from a comparison of an abundance value for the gene or protein in the biological sample and a reference abundance value for the gene or protein. The method further comprises using the differential abundance values of the nodes of the biological interaction network, performing a hill-climbing algorithm to partition the biological interaction network into clusters. The method further comprises determining, from the topology of the clusters, a signature of a molecular phenotype of the biological sample. The method further comprises comparing the signature with a reference signature of a known molecular phenotype.

Advantageously, such a method enables one to determine whether the biological sample in question exhibits the known molecular phenotype associated with the reference signature. For example, the signature can be compared with a known signature representative of a disease phenotype to determine whether the tested sample also exhibits that disease phenotype.

The comparison may be with respect to a database/library of reference signatures, each reference signature corresponding to a known molecular phenotype.

As explained above, if the representative abundance values/expression values of genes or proteins in the biological sample of interest are similar to the reference values, then the resulting clusters may be large and few. However, if the representative values and reference values are significantly different for one or more genes or proteins in the network, then the resulting clusters may be smaller and more plentiful. This will be reflected in the determined signature.

One may repeat such a method herein for determining a molecular phenotype of a biological sample by, using several different sets of reference values. For example, one may derive differential abundance values by comparing the sample to a healthy reference, and the resulting topological signature may indicate that the sample is not healthy, and the pathways/subpathways may indicate that the phenotype belongs to a family of diseases. The algorithm may then be repeated with different differential abundance values derived by comparing the sample with a reference associated with that family of diseases to home in on the correct diagnosis.

According to an aspect of the invention, an apparatus is provided for determining a molecular phenotype of a biological sample. The apparatus comprises one or more memory devices configured to store a biological interaction network. The biological interaction network comprises a plurality of nodes, each node associated with a corresponding gene or protein. The biological interaction network further comprise a plurality of edges, each edge connecting a pair of nodes and indicative of an interaction between the genes or proteins to which each node of that associated pair of nodes corresponds. The apparatus further comprises one or more processors. The one or more processors are configured to associate, with each node of the biological interaction network, a corresponding differential abundance value for the gene or protein to which that node corresponds, the differential abundance value derived from a comparison of an abundance value for the gene or protein in the biological sample and a reference abundance value for the gene or protein. The one or more processors are further configured to, using the differential abundance values of the nodes of the biological interaction network, perform a hill-climbing algorithm to partition the biological interaction network into clusters. The one or more processors are further configured to determine, from the topology of the clusters, a signature of a molecular phenotype of the biological sample. The one or more processors are further configured to compare the signature with a reference signature of a known molecular phenotype to determine a molecular phenotype of the biological sample.

According to an aspect of the invention, a computer-readable medium is provided. The computer-readable medium has instructions stored thereon which, when executed by one or more processors, causes a method as described herein to be performed.

A computer program and/or the code/instructions for performing such methods as described herein may be provided to an apparatus, such as a computer, on a computer readable medium or computer program product. The computer readable medium could be, for example, an electronic, magnetic, optical, electromagnetic, infrared, or semiconductor system, or a propagation medium for data transmission, for example for downloading the code over the Internet. Alternatively, the computer readable medium could take the form of a physical computer readable medium such as semiconductor or solid-state memory, magnetic tape, a removable computer diskette, a random access memory (RAM), a read-only memory (ROM), a rigid magnetic disc, and an optical disk, such as a CD-ROM, CD-R/W or DVD.

Many modifications and other embodiments of the inventions set out herein will come to mind to a person skilled in the art to which these inventions pertain in light of the teachings presented herein. Therefore, it will be understood that the disclosure herein is not to be limited to the specific embodiments disclosed herein. Moreover, although the description provided herein provides example embodiments in the context of certain combinations of elements, steps and/or functions may be provided by alternative embodiments without departing from the scope of the invention.

### BRIEF DESCRIPTION OF THE DRAWINGS

Embodiments of the invention will now be described by way of example only, with reference to the accompanying figures, in which:

FIG. 1 shows a flow chart of a method for characterising a molecular phenotype of a biological sample exhibiting the molecular phenotype;

FIG. 2 shows a flow chart of a hill-climbing algorithm;

FIG. 3 shows a biological interaction network, with associated differential abundance values;

FIG. 4 shows the biological interaction network of FIG. 3 with arrows indicating gradients;

FIG. 5 shows clusters after performing the hill-climbing algorithm on the biological interaction network of FIG. 3;

FIG. 6 shows a flow chart of a method for determining a molecular phenotype of a biological sample for which the molecular phenotype is unknown;

FIG. 7 shows a block diagram of an apparatus; and

FIG. 8 shows a graph of sensitivity against 1-specificty for an experiment carried out by the inventors.

Throughout the description and drawings, like reference numerals refer to like parts.

### DESCRIPTION

Whilst various embodiments are described below, the invention is not limited to these embodiments, and variations of these embodiments may well fall within the scope of the invention which is to be limited only by the appended claims.

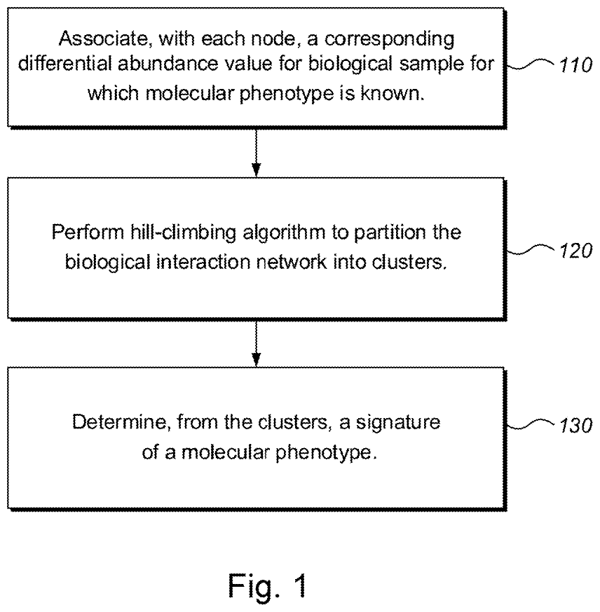

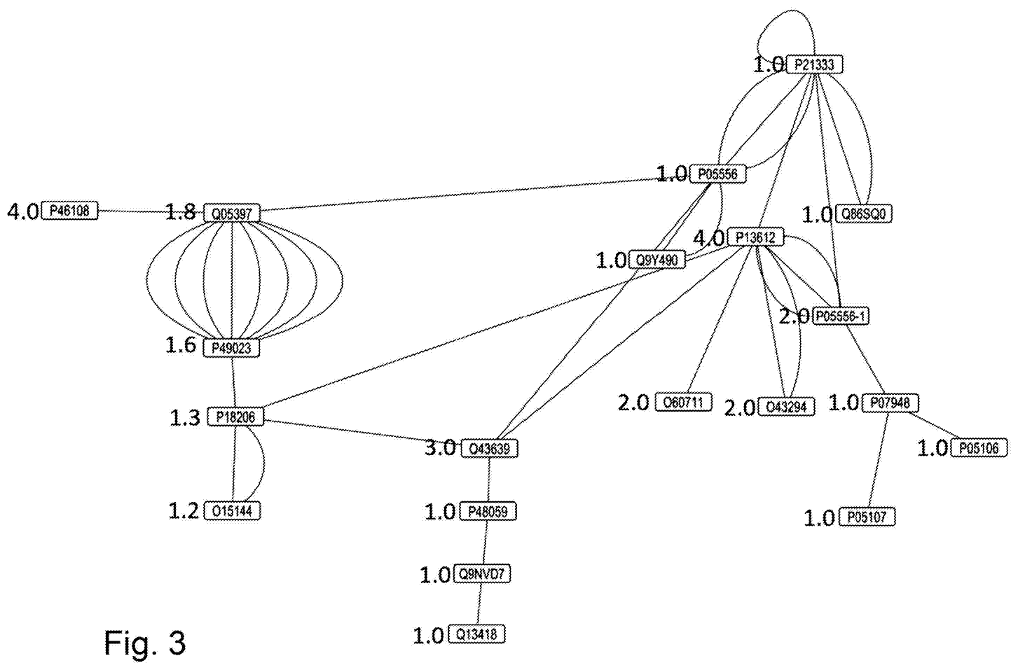

FIG. 1 shows a flowchart of a method for characterising a molecular phenotype of a biological sample using a biological interaction network. The biological interaction network comprises a plurality of nodes, each node associated with a corresponding gene or protein. An example of a biological interaction network is shown in FIG. 3, which in particular shows several (in the example of FIG. 3, twenty) nodes labelled by the white boxes, with each node representing a corresponding protein.

The biological interaction network further comprises a plurality of edges, each edge connecting a pair of nodes and indicative of an interaction between the genes or proteins to which each node of that associated pair of nodes corresponds. The edges may be weighted. For example, in FIG. 3, there is a single line connecting “P49023” and “P18206”, representing an edge weighting of 1, while there are eight lines connecting “P49023” and “QO5397”, representing an edge weight of 8. Such weightings of the edges represent known or hypothesised relative “strengths” of interaction between the proteins identified by the nodes to which the edges correspond.

Referring again to FIG. 1, at step 110 the method comprises associating, with each node of the biological interaction network, a corresponding differential abundance value for the gene or protein to which that node corresponds. The differential abundance value is derived from a comparison of a representative abundance value for the gene or protein in a biological sample exhibiting the molecular phenotype and a reference abundance value for the gene or protein.

In FIG. 3, for which the nodes correspond to proteins, the differential abundance values comprise differential protein abundance/expression values. In particular, the nodes' differential abundance values comprise fold change of each protein abundance in a test patient sample relative to average of each healthy control patient protein. The differential gene expression values are indicated by the numbers next to the nodes in FIG. 3—for example, “1.8” is the differential protein abundance value associated with “QO5397” in FIG. 3.

The differential expression value may be derived from a comparison of the a representative protein abundance value for the relevant protein in a biological sample exhibiting the molecular phenotype with a reference abundance value for the protein. For example, where the molecular phenotype represents a disease state, a representative protein abundance value may comprise an average over the protein abundance values for that protein in patients known to be afflicted with that disease state. For example, the reference abundance value may comprise an average over protein abundance values for that protein in a large group of people, the large group including members of the population that are afflicted with the disease state and members that are not afflicted with the disease state.

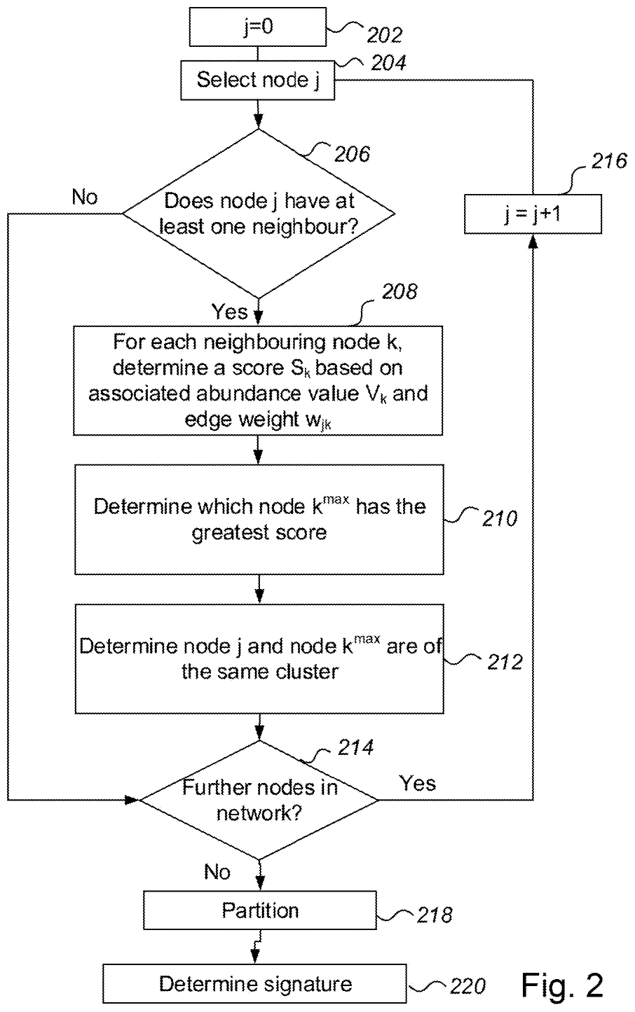

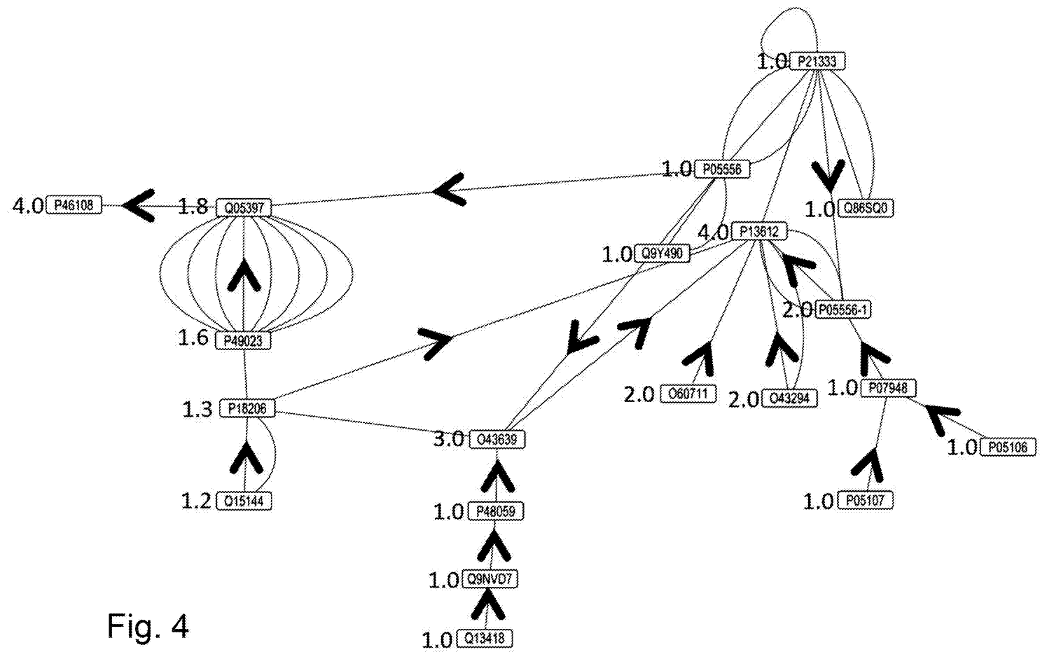

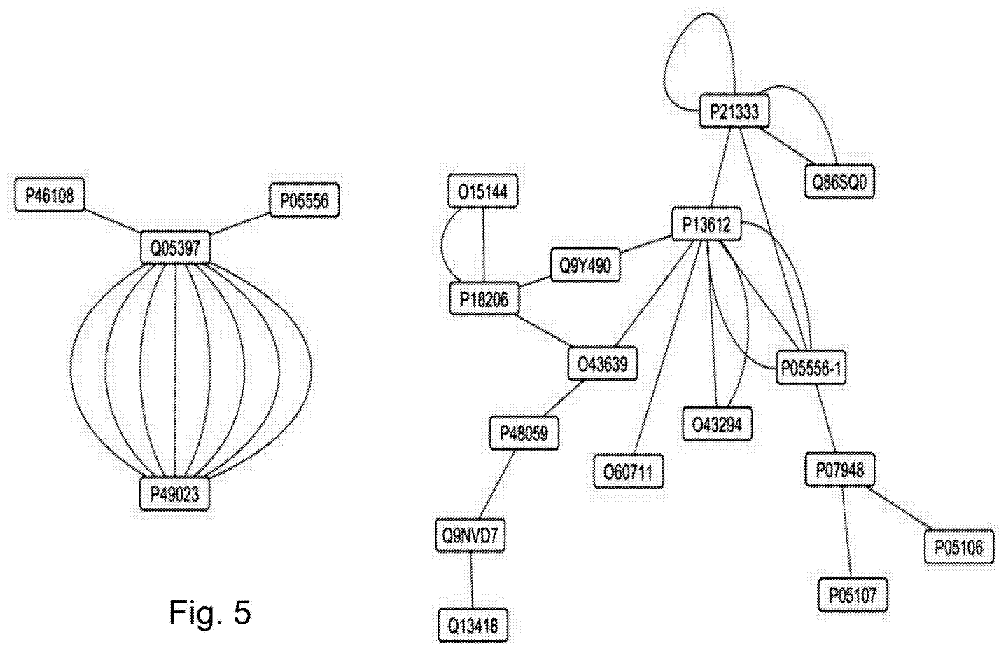

At step 120 in FIG. 1, the method comprises, using the differential abundance values of the nodes of the biological interaction network, performing a hill-climbing algorithm to partition the biological interaction network into clusters. FIG. 4 illustrates the biological interaction network of FIG. 3, with arrows indicating a steepest gradient between nodes. FIG. 5 illustrates the clusters after the hill-climbing algorithm is performed. An example of a hill climbing algorithm will be discussed further below in relation to FIG. 2.

At step 130 in FIG. 1, the method further comprises determining, from the topology of the clusters, a signature of the molecular phenotype. The signature may be any suitable signature, for example the size of the largest cluster and/or the number of clusters.

FIG. 2 shows a flowchart of a hill climbing algorithm that may be used to partition a biological interaction network into clusters, the topologies of which can be used to determine a signature of a molecular phenotype. The algorithm traverses the nodes of the biological interaction network, determining which nodes are of the same cluster (and thereby determining how to partition the network). The nodes can be arbitrarily labelled with indices j=0, 1, 2, . . . , N−2, N−1, where N is the number of nodes in the network.

At step 202, the method is initialised and in this example, j is initialised as zero. At step 204, node j is selected.

At step 206, a determination is made as to whether node j has at least one neighbour. That is, a determination is made as to whether node j is connected to at least one other node via an edge. If a determination is made that node j does not have at least one neighbour (that is, that node j is not connected to any other node via an edge), then the method proceeds to step 214. However, if a determination is made that node j has at least one neighbour then the method proceeds to step 208.

The one or more nodes neighbouring node j can be arbitrarily labelled with indices k=0, 1, 2, . . . , Pⱼ−2, Pⱼ−1, where Pⱼ is the number of nodes neighbouring node j. At 208, for each node k, a score Sₖ is determined. The score Sₖ is based on the differential abundance value Vₖ associated with node k. The score Sₖ is also based on the weight wⱼₖ of the edge connecting node j with node k. The skilled person would appreciate that although the edges may be weighted in this example, in other examples they may not be. In the present example, the edges may be considered as unweighted if all weights are equal, for example wⱼₖ=1 for all nodes j and their corresponding neighbouring nodes k.

The score Sₖ may be determined by any suitable function of the weight wⱼₖ and the differential abundance value Vₖ. For example, the score Sₖ may in some embodiments be defined as the product of the weight with the differential abundance value (wⱼₖVₖ).

The score Sₖ may also be based on the differential abundance value Vⱼ of node j. For example, the score may be determined based on the difference between the differential expression values of nodes j and k, that is on the difference in fold change. For example, the score Sₖ may in some embodiments be defined as the product of the edge weight with the difference in differential abundance values (w^{jk}|Vⱼ−Vₖ| where |x| is the absolute value of x). The score Sₖ may be based on any suitable function of the differential abundance values and weights.

At step 210 a determination is made as to which node k^{max} out of the nodes k neighbouring node j has the greatest score Sₖ^{max}.

Accordingly, at step 212 a determination is made that node j and node k^{max} are of the same cluster. That is, after the hill-climbing algorithm is completed, the node j and node k^{max} will still be connected. The connection between node j and node k^{max} is thus deemed to be in some way “important”.

At step 214, if there are further nodes in the biological interaction network (that is, counter index j has not yet reached N−1), then the index is incremented by one (step 216) and the method returns to step 204 to evaluate the next node. If, at 214, there are no further nodes in the network, then the method proceeds to step 218.

At step 218, the biological interaction network is partitioned into clusters according to the identified associations between nodes. By cycling through steps 204-214 for all nodes in the network, determinations are made (at step 212 in each cycle) as to which nodes should be determined to be part of the same cluster—that is, in each cycle that reaches step 212, a determination is made that node j and node k^{max} are of the same cluster—i.e. should be connected after the partitioning process. Partitioning the network may therefore comprise analysing such established relationship data to determine which connections in the network can be cut. The result is a partitioning of the network into clusters. Every node of the cluster is connected to every other node of that cluster either directly by an “important” edge, or indirectly by a chain of two or more “important” edges. If two nodes are not connected, directly or indirectly, by one or more “important” edges, then the two nodes may be determined to belong to different clusters.

At 220 a signature is determined from the topology of the clusters.

The signature may comprise, for example, an indication as to which nodes of the original biological interaction network belong to the same cluster. For example, the signature may comprise a collection of one or more sets (e.g. one or more vectors or arrays) of node indices, the node indices of each array corresponding to nodes determined to be within the same cluster. The signature may accordingly comprise a collection of disjoint sets, each disjoint set representing a cluster. Such a signature may be thought of as a qualitative signature of the molecular phenotype.

Additionally or alternatively, the signature may comprise a quantitative signature, derived from a calculation performed based on the differential abundance values of the nodes of each cluster. The signature may comprise for example a function of the sizes of the clusters, or the size of the largest cluster.

The skilled person would appreciate that FIG. 2 is an example only and that other such methods may also be applicable—the scope of the invention is to be limited only by the scope of the claims.

For example, if at step 206, if it is determined that there is only one neighbouring node connected to node j (that is, Pⱼ=1 for node j) then it may be determined automatically that node j and that neighbouring node are of the same cluster, and then the method can move on to 214 without needing to pass through steps 208-212.

As an example, the flowchart of FIG. 2 will be described also with reference to FIG. 4, although the skilled person would appreciate that this is for clarity and not intended to be in any way limiting. The hill-climbing algorithm traverses the nodes of the biological interaction network which for the purposes of discussion this discussion are indexed as j=0, 1, 2, . . . , 18, 19. The particular permutation of which indices are associated with each node is unimportant for the purposes of this discussion. However, for the purposes of discussion node 0 corresponds to “O15144” in FIG. 4, and node 1 corresponds to “P18206”.

The method begins at step 202, at which j is initialised, in this case as zero, and at step 204 node 0 (“O15144”) is selected.

At step 206, a determination is made as to whether node 0 has at least one neighbour, that is, a determination is made as to whether node 0 is connected to at least one other node by an edge. In FIG. 4, “O15144” is connected to “P18206” and so the method proceeds to step 208. As “P18206” is the only neighbouring node, that neighbouring node is identified as k^{max}. That is, the connection between “P18206” and “O15144” is deemed to be in some way “important”.

In FIG. 4, the edges labelled with dark arrows are those deemed to be “important”. For example, the weighted edge between “P18206” and “O15144” is one such edge. The direction of the dark arrows in FIG. 4 indicates the gradient (i.e. the direction of a node having the highest differential abundance value of the two nodes that the edge connects).

As explained above, in variations of the method of FIG. 2, as there is only one neighbouring node (“P18206”) for “O15144” the method may bypass steps 208-212 and a determination may be made by default that “O15144” and “P18206” are of the same cluster.

At 212, it is determined that “O15144” and “P18206” belong to the same cluster. The method then proceeds to step 214 and then to step 216. At 216, j is updated such that j=1. Accordingly, at 204, “P18206” is selected.

“P18206” is connected to each of “O15144”, “P49023”, “O43639” and “Q9Y490” and so at step 206 the method proceeds to step 208.

At 208, for each of the neighbouring nodes a score is determined based on the differential abundance values and edge weights of each node. For example, the score can be determined from wⱼₖ|Vⱼ−Vₖ|.

The weight of the edge between “O15144” and “P18206” is 2 (that is, in FIG. 4 there are two lines connecting the two nodes) and so the score for “O15144” is 0.2. The weight of the edge between “P49023” and “P18206” is 1 and so the score for “P49023” is 0.3. Similarly, the score for “O43639” is 1.7 and the score for “Q9Y490” is 0.3. Accordingly, at step 210 it is determined that the node associated with “O43639” has the greatest score.

At step 212, it is determined that “P18206” and “O43639” belong to the same cluster. That is, the connection between “P18206” and “O43639” is deemed to be important, as indicated by the dark arrow connecting these two nodes in FIG. 4. Accordingly, the method has thus far determined that “O15144”, “P18206” and “O43639” are of the same cluster.

At step 214, the method proceeds to step 216 and the method continues until all nodes have been evaluated.

After the hill-climbing algorithm is run, the biological interaction network is partitioned into two clusters as shown in FIG. 5. In comparison to FIG. 4, one can see that only “unimportant” edges have been removed in partitioning the network into clusters, and that each node of a cluster is connected to each other node of a cluster by one or more edges that were deemed important. For example, “P49023” is directly connected to “QO5397”, and “P49023” is indirectly connected to “P46108” (via the connection to “QO5397” and the connection between “QO5397” and “P46108”. Furthermore, and as can be seen by comparing FIG. 5 to FIG. 4, in partitioning the network into clusters the edges that have been removed (or weighted as zero) are edges that connect nodes that are not connected via an alternative path formed of “important” edges.

The topological properties of the clusters of FIG. 5 can be used to generate a signature for the molecular phenotype represented by the differential abundance values. Accordingly, methods according to FIGS. 1 and/or 2 can be used to generate a plurality of signatures of molecular phenotypes, which can be stored in a database (for example) for subsequent use in determining a molecular phenotype of a biological sample for which the molecular phenotype is unknown.

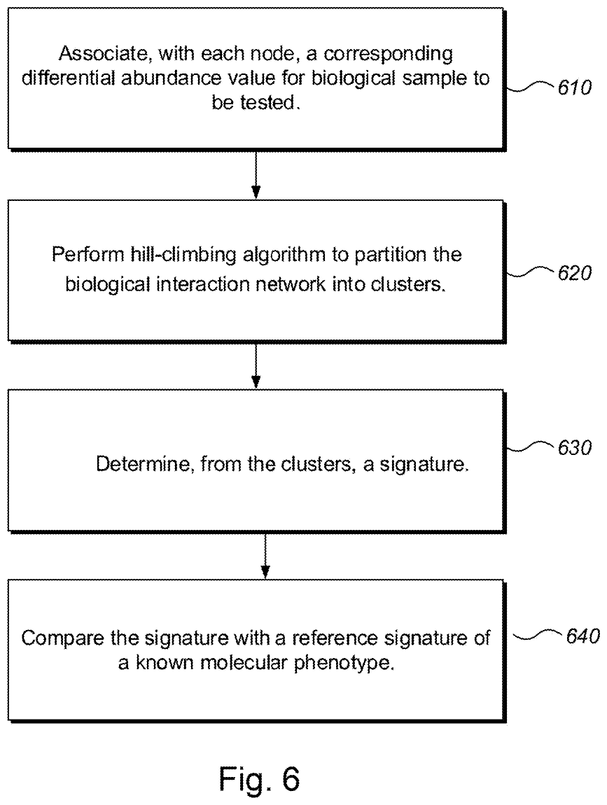

FIG. 6 shows a flowchart of a method for determining a molecular phenotype of a biological sample using a biological interaction network, the biological interaction network comprising a plurality of nodes, each node associated with a corresponding gene or protein, and a plurality of edges, each edge connecting a pair of nodes and indicative of an interaction between the genes or proteins to which each node of that associated pair of nodes corresponds.

At 610, the method comprises associating, with each node of the biological interaction network, a corresponding differential abundance value for the gene or protein to which that node corresponds, the differential abundance value derived from a comparison of an abundance value for the gene or protein in the biological sample and a reference abundance value for the gene or protein.

At 620, the method comprises, using the differential abundance values of the nodes of the biological interaction network, performing a hill-climbing algorithm to partition the biological interaction network into clusters.

At 630, the method comprises determining, from the topology of the clusters, a signature of a molecular phenotype of the biological sample.

At 640, the method comprises comparing the signature with a reference signature of a known molecular phenotype.

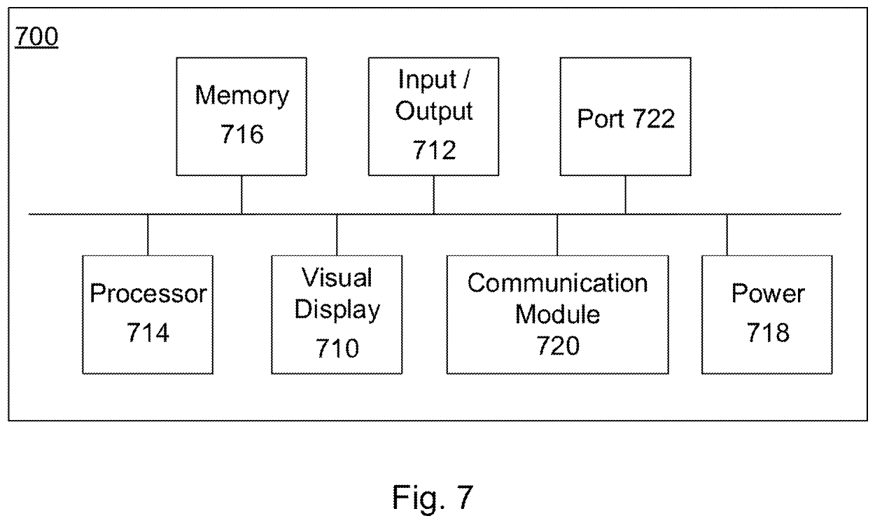

FIG. 7 is a block diagram of a computing apparatus 700. The apparatus/data processing system 700 is an example of a computer, in which computer usable program code or instructions implementing the processes may be located and acted upon. For example, computing apparatus 700 may comprise a computing device, a server, a mobile or portable computer or telephone and so on. Computing apparatus 700 may be distributed across multiple connected devices. Other architectures to that shown in FIG. 7 may be used as will be appreciated by the skilled person. Computing apparatus 700 may be configured to perform the methods of FIGS. 1, 2, and/or 4.

The apparatus 700 includes a number of user interfaces including visualising means such as a visual display 710 and a virtual or dedicated user input/output unit 712. Input/output unit 712 allows for input and output of data with other devices/users that may be connected to apparatus 700. For example, input/output unit 712 may provide a connection for user input through a keyboard, a mouse, and/or some other suitable input device. Further, input/output unit 712 may send output to a printer.

The apparatus 700 further includes one or more processors 714, one or more memory units 716, and a power system 718.

The apparatus 700 comprises a communications module 720 for sending and receiving communications between processor 714 and remote systems. For example, communications module 720 may be used to send and receive communications via a network such as the Internet. Communications module 720 may provide communications through the use of either or both physical and wireless communications links.

The apparatus further comprises a port 722 for receiving, for example, a non-transitory computer-readable medium containing instructions to be processed by the processor 714.

Memory 716 may comprise one or more storage devices such as random access memory or persistent storage. A storage device is any piece of hardware that is capable of storing information, such as, for example, without limitation, data, program code in functional form, and/or other suitable information either on a temporary basis and/or a permanent basis. Memory 716, in these examples, may be, for example, a random access memory or any other suitable volatile or non-volatile storage device. Memory units for persistent storage may take various forms depending on the particular implementation. For example, persistent storage may contain one or more components or devices. For example, persistent storage may be a hard drive, a flash memory, a rewritable optical disk, a rewritable magnetic tape, or some combination of the above. The media used by persistent storage also may be removable. For example, a removable hard drive may be used for persistent storage.

Instructions for the processor 714 may be stored. For example, the instructions may be in a functional form in persistent storage of the one or more memory units 716. These instructions may be loaded into active (e.g. random access) memory for execution by processor 714.

Processor 714 serves to execute instructions for software that may be loaded into memory 716. Processor unit 714 may be a set of one or more processors or may be a multiprocessor core, depending on the particular implementation. Further, processor unit 714 may be implemented using one or more heterogeneous processor systems in which a main processor is present with secondary processors on a single chip. As another illustrative example, processor unit 714 may be a symmetric multi-processor system containing multiple processors of the same type.

The processor 714 is configured to receive data, access the memory 716, and to act upon instructions received from said memory 716, from communications module 720 or from user input device 712.

The computing apparatus 700 may used to characterise a molecular phenotype of a biological sample. Data representative of a biological interaction network may be stored at least in part in memory 716 and/or received at least in part via communications module 720.

The processor 714 may be configured to associate, with each node of the biological interaction network, a corresponding differential abundance value for the gene or protein to which that node corresponds, the differential abundance value derived from a comparison of a representative abundance value for the gene or protein in a biological sample exhibiting the molecular phenotype and a reference abundance value for the gene or protein. The processor may derive the differential abundance values itself from stored or received data or may receive the differential abundance values from an external source.

The processor 714 may be configured to, using the differential abundance values of the nodes of the biological interaction network, perform a hill-climbing algorithm to partition the biological interaction network into clusters.

The processor 714 may be configured to determine, from the topology of the clusters, a signature of the molecular phenotype. The processor may be further configured to communicate the signature via the input/output unit 712, the visual display 710, or he communication module 720, and/or may store the signature in memory 716.

The computing apparatus 700 may be used to determine of determining a molecular phenotype of a biological sample using a biological interaction network. Data representative of a biological interaction network may be stored at least in part in memory 716 and/or received at least in part via communications module 720.

The processor 714 may be configured to associate, with each node of the biological interaction network, a corresponding differential abundance value for the gene or protein to which that node corresponds, the differential abundance value derived from a comparison of an abundance value for the gene or protein in the biological sample and a reference abundance value for the gene or protein.

The processor may be configured to, using the differential abundance values of the nodes of the biological interaction network, perform a hill-climbing algorithm to partition the biological interaction network into clusters.

The processor 714 may be configured to determine, from the topology of the clusters, a signature of a molecular phenotype of the biological sample.

The processor may be configured to compare the signature with a reference signature of a known molecular phenotype. For example, the memory 716 may contain a database/library of signatures corresponding to known molecular phenotypes, against which a signature may be compared. The processor my communicate the signature and/or a determination of the molecular phenotype via the input/output unit 712, the visual display 710, or he communication module 720, and/or may store the signature in memory 716.

As an example of the methods described herein, the inventors have used publicly available global expression data (microarray) from blood peripheral blood mononuclear cell (PBMC) samples from patients with different disease diagnoses to identify disease state phenotypes.

In particular, the inventors downloaded the STRING protein interaction network available at https://string-db.org/cgi/download.pl?sessionid=PLOH2DmpLf8W.

A reference biological interaction network was created for which protein (gene) nodes were annotated with the mean average abundances of each gene across a large number of patients. That is, for each gene associated with a node of the network, a reference abundance value was derived from the average of the expression values for that gene in a large number of patients.

For each patient, individually, fold change gene expression was calculated relative to the reference network, and fold change gene expression data was applied as metadata to protein (gene) nodes in the network.

The network was partitioned according to network topology—a custom Morse theory algorithm was used to calculate the flow of differential gene modulation throughout the network, along edges defined in the gene network. This defines clusters of differentially modulated genes in a patient relative to the reference, and therefore identifies differentially modulated pathways, and possible upstream regulators responsible for the modulation of the pathway(s).

For each disease group in the database, the average disease patient was calculated and the corresponding gene clusters were produced.

For each patient, the normalized mutual information (NMI) score was calculated with each of the disease model patients. The mutual information is a measure of the similarity between two labels of the same data. Where |Aᵢ| is the number of samples in cluster Aᵢ, and |Bⱼ| is the number of samples in cluster Bⱼ, the mutual information between clusterings A and B is given as:

$$MI ( A , B ) = {\sum }_{i = 1}^{\vert A \vert} {\sum }_{j = 1}^{\vert B \vert} \frac{\vert {A}_{i} ⋂ {B}_{j} \vert}{Z} log \frac{Z \vert {A}_{i} ⋂ {B}_{j} \vert}{\vert {A}_{i} \vert \vert {B}_{j} \vert}$$

where Z is the number of objects (i.e. nodes) in the clustering. The normalized mutual information is the mutual information divided by the mean of the Shannon entropy of the two sets A and B.

The inventors plotted a multilabel classification received operating characteristic (ROC) curve, where a patient closeness to a disease model was given by the NMI score.

The accuracy was determined with an ROC Area-Under-Curve (AUC) of 0.97, representing very good classification accuracy (molecular diagnosis).

In particular, the inventors were able to accurately and precisely diagnose 8 different diseases in 239 patients using blood microarray gene expression data from Chaussabel et al., 2008. A modular analysis framework for blood genomics studies: application to systemic lupus erythematosus. Immunity, 29(1), pp. 150-164.

239 peripheral blood mononuclear cell (PBMC) samples obtained from individuals:
Systemic juvenile idiopathic arthritis (n=47)
Systemic lupus erythematosus (n=40)
Type I diabetes (n=20)
Metastatic melanoma (n=39)
Acute infections (Escherichia coli (n=22)
Staphylococcus aureus (n=18)
Influenza A (n=16)
Liver transplant recipients undergoing immunosuppressive therapy (n=37)
Transcriptional profiles were generated using Affymetrix U133A and U133B GeneChips (>44,000 probesets)

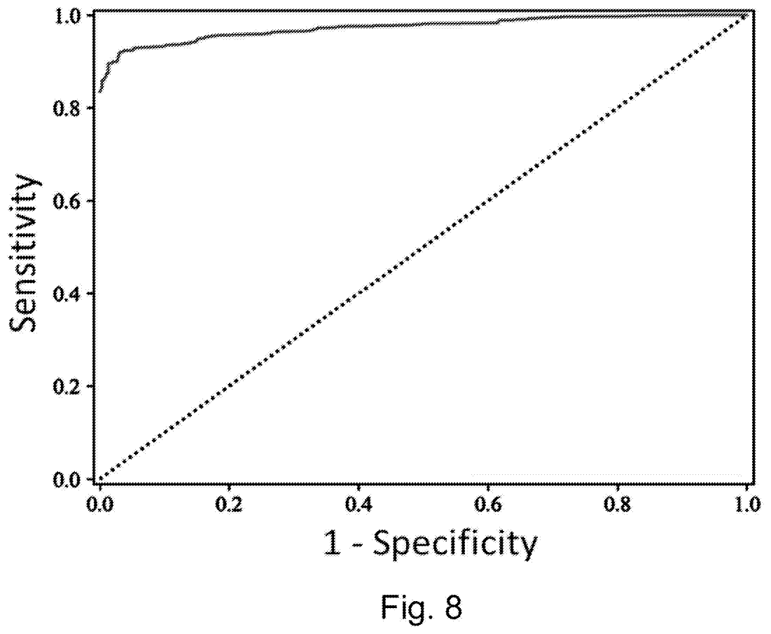

FIG. 8 shows a graph of Sensitivity plotted against 1-Specificity of the test described above in classifying patient phenotypes by similarity in clusters. The graph demonstrates the high accuracy and precision of the techniques described herein in classifying a diversity of molecular phenotypes from similarly acquired gene expression profiling data.

It will be appreciated that embodiments of the present invention can be realised in the form of hardware, software or a combination of hardware and software. Any such software may be stored in the form of volatile or non-volatile storage such as, for example, a storage device like a ROM, whether erasable or rewritable or not, or in the form of memory such as, for example, RAM, memory chips, device or integrated circuits or on an optically or magnetically readable medium such as, for example, a CD, DVD, magnetic disk or magnetic tape. It will be appreciated that the storage devices and storage media are embodiments of machine-readable storage that are suitable for storing a program or programs that, when executed, implement embodiments of the present invention. Accordingly, embodiments provide a program comprising code for implementing a system or method as claimed in any preceding claim and a machine-readable storage storing such a program. Still further, embodiments of the present invention may be conveyed electronically via any medium such as a communication signal carried over a wired or wireless connection and embodiments suitably encompass the same.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed. The claims should not be construed to cover merely the foregoing embodiments, but also any embodiments which fall within the scope of the claims.

1. A computer-implemented method of characterizing a molecular phenotype of a biological sample using a biological interaction network, the biological interaction network comprising a plurality of nodes, each node associated with a corresponding gene or protein, the method comprising:
associating, with each node of the biological interaction network, a corresponding differential abundance value for the gene or protein to which that node corresponds, the differential abundance value derived from a comparison of a representative abundance value for the gene or protein in a biological sample exhibiting the molecular phenotype and a reference abundance value for the gene or protein;
using the differential abundance values of the nodes of the biological interaction network, performing a hill-climbing algorithm to partition the biological interaction network into clusters; and
determining, from the topology of the clusters, a signature of the molecular phenotype.

2. The method according to claim 1, wherein the biological interaction network comprises a plurality of edges, each edge connecting a pair of nodes and indicative of an interaction between the genes or proteins to which each node of that associated pair of nodes corresponds, the method further comprising:
associating, with each edge of the plurality of edges, a weight; and
wherein performing the hill-climbing algorithm comprises performing the hill-climbing algorithm using the weights of the edges.

3. The method according to claim 1,
wherein each node of the plurality of nodes is associated with a corresponding gene;
wherein the representative abundance value for the gene comprises a gene expression value for the gene;
wherein the reference abundance value for the gene comprises a reference gene expression value for the gene; and
wherein the differential abundance value comprises a differential gene expression value, the differential gene expression value derived from a comparison of the representative gene expression value and the reference gene expression value.

4. The method according to claim 1, wherein the molecular phenotype of the biological sample comprises a disease state of the biological sample.

5. The method according to claim 1, wherein the biological network comprises a biological pathway.

6. The method according to claim 1, further comprising, prior to the associating, receiving data representative of the biological interaction network.

7. The method according to claim 1, further comprising, for each node of the biological network, receiving or determining the corresponding differential abundance value.

8. The method according to claim 1, wherein the reference abundance value for each node comprises an average of abundance values for a plurality of biological samples.

9. The method according to claim 1, wherein the representative abundance value for each node comprises an average of abundance values for a plurality of biological samples exhibiting the molecular phenotype.

10. The method according to claim 1, wherein performing the hill-climbing algorithm comprises performing a Morse theory algorithm.

11. The method according to claim 1, wherein performing the hill-climbing algorithm to partition the biological interaction network into clusters comprises:
for each node of the biological interaction network,
determining, for each neighboring node of all neighboring nodes connected to the node, a score based on the differential abundance value of the neighboring node;
determining, out of all neighboring nodes connected to the node, the neighboring node associated with the highest or lowest score; and
determining that the node and the neighboring node associated with the highest or lowest score are of the same cluster.

12. A computer-readable medium having instructions stored thereon which, when executed by a processor, causes a method according to claim 1 to be performed.

13. An apparatus for characterizing a molecular phenotype of a biological sample, the apparatus comprising:
one or more memory devices configured to store a biological interaction network, the biological interaction network comprising:
a plurality of nodes, each node associated with a corresponding gene or protein; and
a plurality of edges, each edge connecting a pair of nodes and indicative of an interaction between the genes or proteins to which each node of that associated pair of nodes corresponds; and
one or more processors configured to:
associate, with each node of the biological interaction network, a corresponding differential abundance value for the gene or protein to which that node corresponds, the differential abundance value derived from a comparison of a representative abundance value for the gene or protein in a biological sample exhibiting the molecular phenotype and a reference abundance value for the gene or protein;
using the differential abundance values of the nodes of the biological interaction network, perform a hill-climbing algorithm to partition the biological interaction network into clusters; and
determine, from the topology of the clusters, a signature of the molecular phenotype.

14. A computer-implemented method of determining a molecular phenotype of a biological sample using a biological interaction network, the biological interaction network comprising a plurality of nodes, each node associated with a corresponding gene or protein;
the method comprising:
associating, with each node of the biological interaction network, a corresponding differential abundance value for the gene or protein to which that node corresponds, the differential abundance value derived from a comparison of an abundance value for the gene or protein in the biological sample and a reference abundance value for the gene or protein;
using the differential abundance values of the nodes of the biological interaction network, performing a hill-climbing algorithm to partition the biological interaction network into clusters;
determining, from the topology of the clusters, a signature of a molecular phenotype of the biological sample; and
comparing the signature with a reference signature of a known molecular phenotype.

15. An apparatus for determining a molecular phenotype of a biological sample, the apparatus comprising:
one or more memory devices configured to store a biological interaction network, the biological interaction network comprising:
a plurality of nodes, each node associated with a corresponding gene or protein; and
a plurality of edges, each edge connecting a pair of nodes and indicative of an interaction between the genes or proteins to which each node of that associated pair of nodes corresponds; and
one or more processors configured to:
associate, with each node of the biological interaction network, a corresponding differential abundance value for the gene or protein to which that node corresponds, the differential abundance value derived from a comparison of an abundance value for the gene or protein in the biological sample and a reference abundance value for the gene or protein;
using the differential abundance values of the nodes of the biological interaction network, perform a hill-climbing algorithm to partition the biological interaction network into clusters;
determine, from the topology of the clusters, a signature of a molecular phenotype of the biological sample; and
compare the signature with a reference signature of a known molecular phenotype to determine a molecular phenotype of the biological sample.

16. A computer-readable medium having instructions stored thereon which, when executed by a processor, causes the method according to claim 14 to be performed.

17. The computer-implemented method of claim 14, wherein the biological interaction network comprises a plurality of edges, each edge connecting a pair of nodes and indicative of an interaction between the genes or proteins to which each node of that associated pair of nodes corresponds.

18. The apparatus of claim 13, the one or more processors further configured to associate, with each edge of the plurality of edges, a weight, wherein when performing the hill-climbing algorithm, the one or more processors are to perform the hill-climbing algorithm using the weights of the edges.

19. The apparatus of claim 13, wherein the molecular phenotype of the biological sample comprises a disease state of the biological sample.

20. The apparatus of claim 13, wherein the biological network comprises a biological pathway.
